(19) United States
(12) Patent Application Publication
(10) Pub. No.: US 2002/0128629 A1
(43) Pub. Date: Sep. 12, 2002

(54) Device for connection between a recipient and a container, and ready-to-use assembly comprising such a device

(76) Inventor: Aneas Antoine, Menetrol (FR)

Correspondence Address: MONIQUE A. MORNEAULT, ESQ., WALLENSTEIN & WAGNER, 311 South Wacker Drive - 5300, Chicago, IL, 60606, US

(21) Appl. No.: 10/091,897

(22) Filed: Mar. 6, 2002

(30) Foreign Application Priority Data
Mar. 7, 2001 (FR) 0103125

(51) Int. Cl. A61B 19/00; A61M 5/32

(52) U.S. Cl. 604/414; 604/411

(57) ABSTRACT

This connection device (1) comprises a base (10) adapted to be mounted on the recipient (2) and comprising a sleeve (16) forming an inner bore, and a plunger (20) adapted to slide in said bore, between a first position disengaged with respect to said stopper of the recipient and a so-called position of transfer, in which its hollow needle (21) traverses the stopper. The needle (21) presents a non-circular outer cross-section, while the sleeve (16) presents a likewise noncircular inner cross-section, the outer section of the needle (21) and inner section of the sleeve (16) being such that the needle can slide in the sleeve, without the possibility of rotation of the needle in the sleeve. The edge (16a) of the sleeve opposite said stopper is provided with stop means (16a, 16b) adapted to cooperate with complementary means (22d, 22e) provided on the plunger (20) to lock it in position of transfer.

The present invention relates to a device for connection between a closed recipient and a container. The invention also relates to a ready-to-use assembly comprising, inter alia, a closed recipient and a connection device of the aforementioned type.

In the domain of drug-packaging, it is known to store a component of a pharmaceutical preparation, such as for example its active ingredient, in a recipient closed by a stopper of relatively non-rigid material, for example elastomer. A liquid may be introduced into this recipient, after perforation of the stopper, in order to dissolve the component contained in this recipient or place it in suspension, with a view to obtaining a preparation, in particular a medicament or a vaccine, in liquid form and ready to be administered to the patient.

WO-A-98/13006 discloses a connection device comprising a base adapted to cover the neck of a recipient and extending in a flange or sleeve forming an inner bore while a plunger is mounted to slide in this bore. The plunger bears snap teeth distributed about its axis and provided to cooperate with an annular face of the flange. This device is satisfactory for connecting a syringe provided with a luer-type connector.

However, it is sometimes necessary to connect a recipient to a container other than a syringue, in which case the liquid is transferred between the recipient and the container, or vice versa, by gravity. In that case, a double-tipped needle with channels of relatively large section must be used. Taking into account the volume of this needle, the effort of reaction of the stopper, which tends to push it out of the recipient, is so great that a snap system as disclosed in WO-A-98/13006 is not forcibly sufficient.

It is therefore an object of the present invention to propose a connection device of the type mentioned above in which the structures of the base and the plunger are particularly simple, while the plunger is efficiently guided in translation with respect to the base and efficiently maintained in position of transfer, including in the case when a double-tipped needle is used.

To that end, the invention relates to a device in which the needle of the plunger presents a non-circular outer cross-section, while a sleeve formed by the base presents a likewise non-circular inner cross-section, the outer section of the needle and inner section of the sleeve being such that the needle can slide in the sleeve, without the possibility of rotation of the needle. In addition, the edge of the sleeve opposite the stopper of the recipient is provided with stop means adapted to cooperate with complementary means provided on the plunger to lock it in position of transfer.

Thanks to the invention, the structure of the plunger may be greatly simplified with respect to the devices of the state of the art, since the shape of the needle that it bears or that it comprises allows a guiding in translation in the sleeve, without possibility of rotation. Slide without possibility of rotation guarantees an adequate positioning of the stop means provided on the upper edge of the sleeve and of the complementary means provided on the plunger. Taking into account its particularly simple structure and its small volume, the plunger of the device of the invention is easy to mould, while the cost price of its material is very attractive, and the geometry of the device facilitates the movement of the plunger from its disengaged position to its position of transfer.

According to a first embodiment of the invention, the needle may be provided to present an oval outer cross-section, while the inner cross-section of the sleeve is likewise oval.

According to another embodiment of the invention, the needle may present a polygonal outer cross-section, while the inner cross-section of the sleeve is polygonal, with the same number of sides as the outer section of the needle.

According to advantageous aspects of the invention, the device incorporates one or more of the following characteristics:

The stop means provided on the edge of the sleeve comprise elastically deformable hooks, while the complementary means provided on the plunger comprise bearing surfaces made on a flange in one piece with the needle, the needle and the flange together constituting the plunger. In that case, the flange is advantageously provided with openings for passage of the hooks. The hooks may each be provided with a nose adapted to be imbricated with a return bordering one of the bearing surfaces made on the flange. In addition, the hooks may be provided to project radially towards the outside with respect to the sleeve.

The base comprises a second sleeve disposed radially outside the aforementioned sleeve, the second sleeve being adapted to cooperate with a cap for protecting the plunger with respect to the ambient atmosphere. Rigidifying ribs may be arranged between these sleeves.

Means are provided for temporarily stopping the plunger in the disengaged position of the needle. These means may comprise at least one hollow made on the outer surface of the needle and at least one projection extending, from the inner radial surface of the sleeve, in the direction of a central axis of the bore, the projection being adapted to be engaged in the hollow in order to maintain the plunger in disengaged position with respect to the stopper of the recipient.

The invention also relates to a ready-to-use assembly comprising a closed recipient containing a product, particularly a pharmaceutical preparation, this recipient being provided with a neck whose opening is closed by a stopper, and a connection device as described hereinabove, mounted on this recipient. Such an assembly makes it possible to conserve a component of a medicament or a vaccine, particularly its active ingredient, sterile, and to prepare it when required by mixing with a liquid, this ready-to-use assembly being particularly easy to manoeuvre.

The invention will be more readily understood and other advantages thereof will appear more clearly on reading the following description of two embodiments of a connection device in accordance with its principle, given solely by way of example and made with reference to the accompanying drawings, in which:

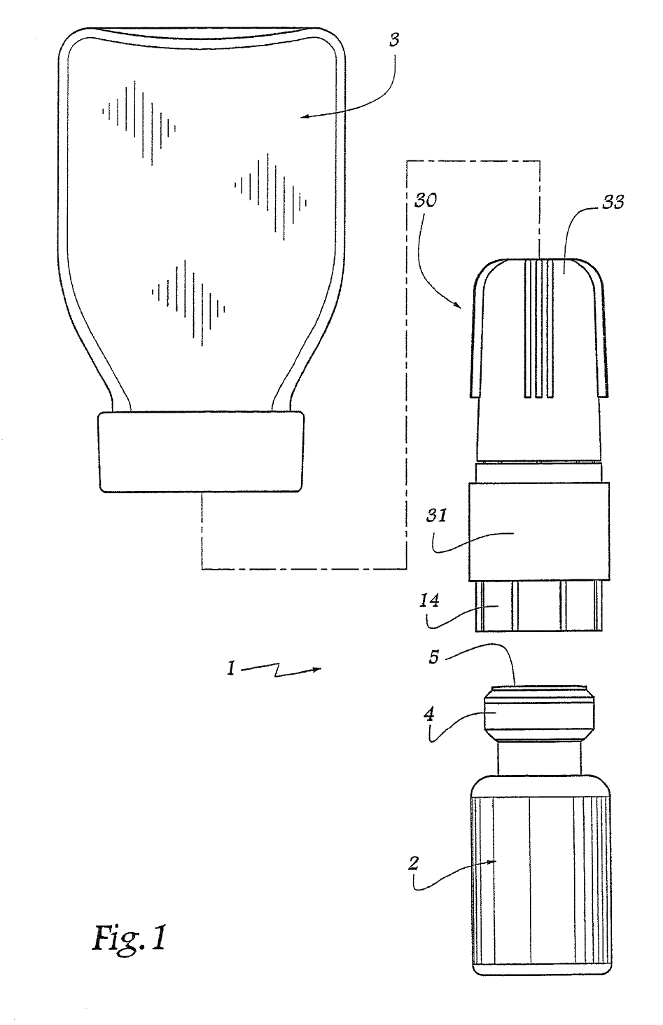

FIG. 1 is an exploded side view of a ready-to-use assembly according to the invention and of a flask provided to cooperate with this assembly.

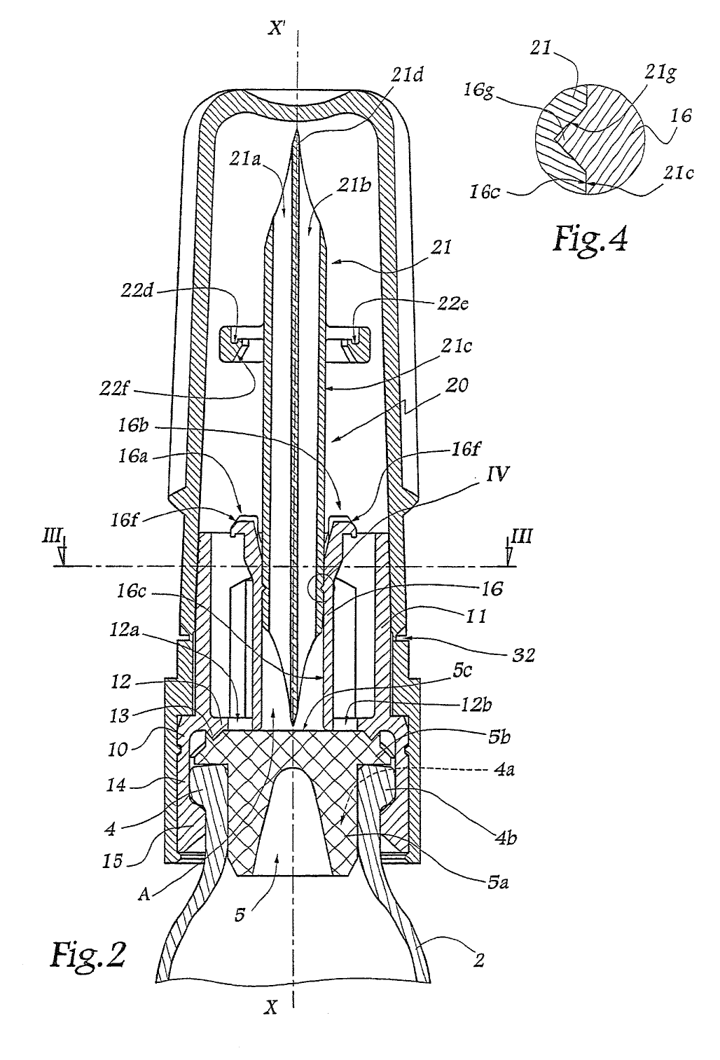

FIG. 2 is an axial section of the connection device belonging to the assembly of FIG. 1, mounted on the recipient in storage configuration.

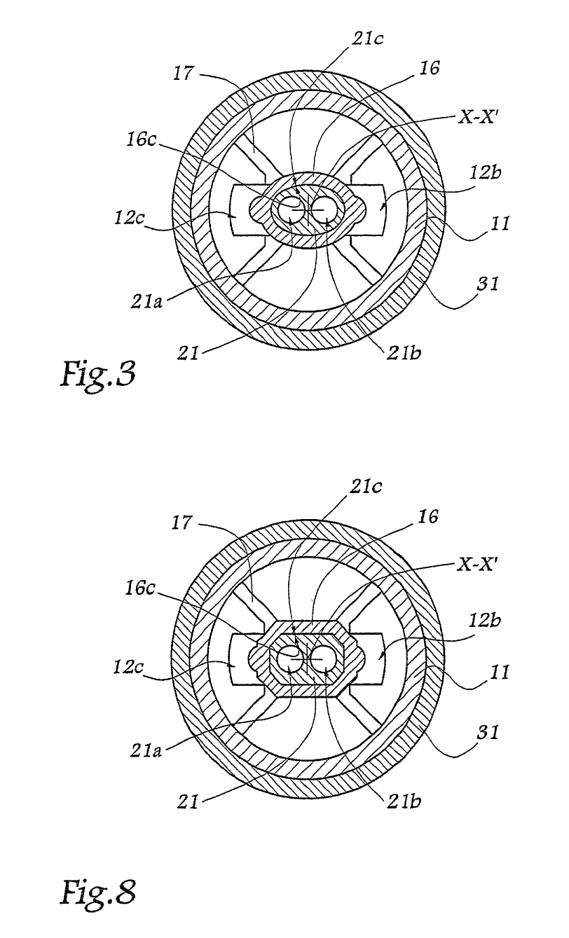

FIG. 3 is a section along line III-III of FIG. 2.

FIG. 4 is a view on a larger scale of detail IV of FIG. 2.

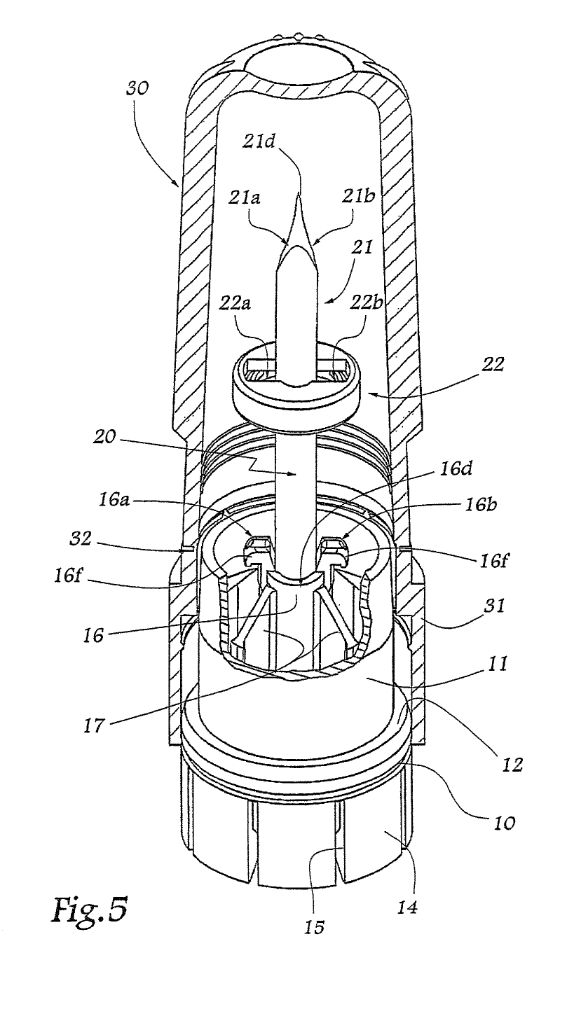

FIG. 5 is a view in perspective, with parts torn away, of the connection device before it is mounted on the recipient.

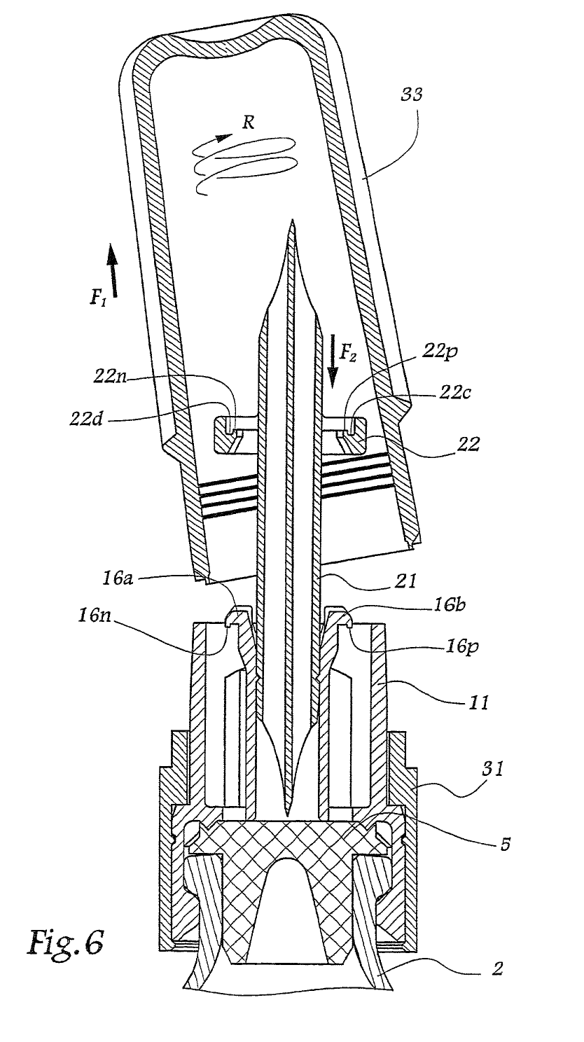

FIG. 6 is a view similar to FIG. 2, at the beginning of an operation for connecting the recipient with the supple flask shown in FIG. 1.

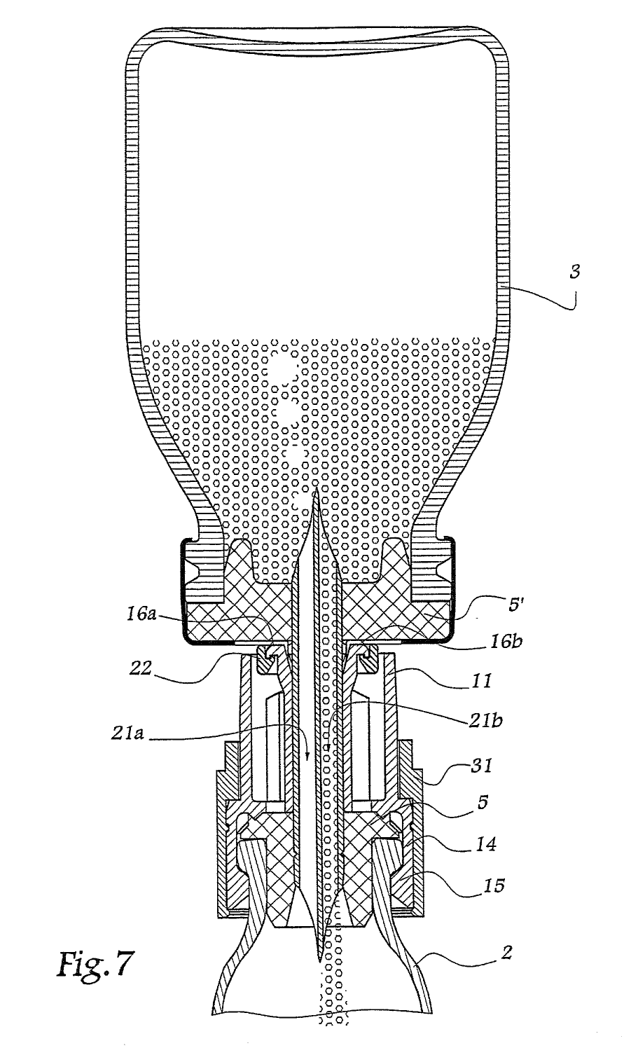

FIG. 7 is a view similar to FIG. 2, while the flask is connected to the recipient of the assembly of FIG. 1, and

FIG. 8 is a section similar to FIG. 3, for a device according to a second embodiment of the invention.

Referring now to the drawings, the device 1 according to the invention shown in FIGS. 1 to 6 has a dual function. On the one hand, it renders tamperproof a recipient 2, for example a glass flask, containing a product (not shown) and previously closed or stoppered. On the other hand, the device 1 ensures or establishes a tight connection beween the interior of the recipient 2 and the interior of another container, such as a flask 3 made of rigid or semi-rigid plastics material containing a liquid intended to dissolve the product contained in the recipient 2, or place it in suspension. The container might also be formed by a glass flask.

The flask 2 comprises a neck 4 whose opening 4a is hermetically closed by a stopper 5 made of a relatively non-rigid material, for example an elastomer, preferably of rubber. According to a variant of the invention (not shown), a capsule may be arranged around the stopper 5 and an outer annular part 4b of the neck 4. The stopper S comprises a substantially cylindrical central part 5a adapted for supple and tight fit inside the opening 4a and a flattened part 5b shouldered on the part 4b and possibly covered by the capsule.

The device 1 comprises a base 10 made by injection of plastics material, for example polyethylene or polypropylene, and intended to be mounted around elements 4 and 5. The base 10 forms a substantially cylindrical sleeve 11 centred on an axis X-X′, which is an axis of symmetry of elements 2, 4 and 5 in mounted configuration as shown in FIG. 2. The sleeve 11 is connected to an annular part 12 provided with a sharp-profiled ring 13 intended to penetrate superficially on the upper or exposed surface Sc of the stopper 5. The annular part 12 extends, opposite the sleeve 11, in a plurality of elastic tabs 14 each provided with a beak 15 adapted to cooperate with the outer surface of the neck 4, with a view to an elastic clipping of the base 10 on the neck.

Inside the sleeve 11, there is formed a second sleeve 16 which extends in the direction of axis X-X′ and presents an oval inner cross-section as shown in FIG. 3.

Sleeves 11 and 16 are connected by rigidifying ribs 17 ensuring adequate positioning and rigidity of the sleeve 16.

The interior volume of the sleeve 16 constitutes a bore A inside which may slide a plunger 20 formed by a piece of moulded plastics material. The plunger 20 comprises a double-tipped hollow needle 21 provided with two channels 21a, 21b and adapted to perforate the stopper 5. The plunger 20 also comprises a flange 22, in one piece with the needle 21 and provided with two openings 22a and 22b.

As is likewise visible in FIG. 3, the outer cross-section of the needle 21 is oval.

21c denotes the outer radial surface of the needle 21, 16c denotes the inner radial surface of the sleeve 16. The surfaces 16c and 21c are such that slide of the needle 21, i.e. of plunger 20, in the bore A is possible. The noncircular nature about axis X-X′ of the surfaces 16c and 21c is such that the plunger 20 may slide in the bore A without the possibility of rotation about axis X-X′ with respect to the sleeve 16.

Two elastic hooks 16a and 16b are provided at the level of the upper edge 16d of the sleeve 16, i.e. the edge of this sleeve opposite the stopper 5. These hooks extend near the two parts of the sleeve of smallest radius of curvature.

The hooks 16a and 16b are configured in order to be able to penetrate in the openings 22a and 22b in order to clip on retaining surfaces 22d and 22e provided in the flange 22.

It will be noted that openings 22a and 22b are each provided with an edge 22f convergent in the direction of the end 21d of the needle 21 opposite the stopper 5. Furthermore, the hooks 16a and 16b are provided with truncated edges 16f convergent opposite the part 12. In this way, the cooperation of the edges 16f and 22f has the effect of tightening the hooks 16 in the direction of axis X-X′ when the flange 22 comes into abutment against these hooks, this allowing the hooks 16a and 16b to pass through the openings 22a and 22b before expanding radially again towards the outside, so that the hooks 16a and 16b come into abutment on the surfaces 22d and 22e.

When the hooks 16a and 16b are in abutment on the surfaces 22d and 22e, the plunger 20 is efficiently maintained in position of transfer of fluid towards or from the interior volume of the recipient 2, this position being shown in FIG. 7.

As is more particularly visible in FIG. 6, the hooks 16a and 16b are each provided with a nose 16n, 16p, respectively, directed towards part 12. Furthermore, the flange 22 is equipped with returns 22n and 22p which respectively border the surfaces 22d and 22e. In this way, when the hooks 16a and 16b are engaged in the openings 22a and 22b, the noses 16n and 16p come into abutment against the surfaces 22d and 22e, to the rear of the returns 22n and 22p. Any risk of relative slide of the hooks 16a and 16b with respect to the flange 22 is thus avoided, as such a slide is prevented by the bearing surface of the noses 16n and 16p against the returns 22n and 22p.

In order to guarantee that the plunger 20 is maintained in configuration of storage of the device 1, i.e. in a configuration in which the needle 21 is at a distance from the stopper 5 as shown in FIGS. 1 to 6, the sleeve 16 is provided with an inner ring 16g projecting from the surface 16c in the direction of axis X-X′. Furthermore, the surface 21c of the needle 21 is provided with a groove 21g in which the ring 16g may be engaged. When the ring 16g is engaged in the groove 21g, the needle 21, i.e. the plunger 20, is immobilized in translation in the bore A.

According to variant embodiments of the invention (not shown), the ring 16g and the groove 21g may be replaced by other elements in relief which may extend over only a part of the circumference of the surfaces 16c and 21c.

An outer envelope 30 is provided to be mounted around the base 10 and the plunger 20 on the recipient 2. The envelope 30 comprises a ring 31 provided to immobilize the tabs 14 in position around the neck 4, in accordance with the technical teaching of WO-A-97/10156. The ring 31 is connected by a breakable section 32 to a cap 33 which protects the base 10 and the plunger 20 from the ambient atmosphere as long as the breakable section has not been broken. It will be noted that the cap 33 is provided to abut against the outer radial surface of the sleeve 11.

A lubricant might be discontinuously applied on the internal surface of the cap 33, in order to facilitate its removal from the base 10. This lubricant could be applied around the sleeve 11, also discontinuously, that is without constituting a continuous circumference around axis X-X′.

Functioning is as follows:

When a liquid is to be made to penetrate inside the recipient 2, the cap 33 is removed by breaking the section 32, as represented by arrows R and F₁ in FIG. 6. It is then possible to exert on the plunger 1 an effort F₂ directed towards the stopper 5 in order to transpierce the latter thanks to the needle 21. In practice, the effort F₂ can be exerted by means of the flask 3 of which a stopper 5′ is firstly engaged on the exposed end 21 c of the needle 21, the stopper 5′ then being able to be held by the user in order to exert on the base 22 the effort F₂, which has the effect of driving the ring 16g from the groove 21g then of allowing the movement of advance of the needle 21 in the direction of the stopper 5 which it then traverses.

During this movement, the hooks 16a and 16b penetrate in the openings 22a and 22b, being deformed thanks to the cooperation of the edges 16f and 22f. The hooks 16a and 16b then lock on the surfaces 22d and 22e of the flange 22, with the result that the plunger 20 is clicked in position of transfer on the sleeve 16.

Any slide of the hooks 16a and 16b with respect to the flange 22, under the effect of an effort tending to extract the needle 21 from the recipient 2, is prevented by the imbrication of the noses 16n and 16p behind the returns 22n and 22p.

During the movement of penetration of the needle 21 in the stopper 5, the sleeve 16 is maintained rigid thanks to the ribs 17 which ensure take-up of any transverse efforts.

When a sufficient quantity of the liquid has been introduced in the recipient 2, it is possible to shake the latter to mix the liquid with the product originally present in the recipient 2 in order to obtain the desired solution or suspension. It is then possible to upturn the assembly formed by elements 1 to 3 in order to pass this solution or suspension into the flask 3 with a view to using it.

The hooks 16a and 16b are obtained without using a complex mould, two openings 12a and 12b being provided in part 12 for the passage of slides for forming these hooks in the mould manufacturing the base 10.

The base 10, comprising elements 11 to 17, is in one piece. The elements constituting the device 1 of the invention are therefore particularly simple and of very attractive cost price.

In the second embodiment shown in FIG. 8, elements similar to those of the first embodiment bear identical references. This embodiment differs from the preceding one in that the needle 21 presents a polygonal outer radial cross-section, while the sleeve 16 has a polygonal inner cross-section, with the same number of sides and the same geometric distribution of these sides, which allows the needle 21 to slide in the sleeve 16 along axis X-X′, without the possibility of rotation about this axis.

Whatever the embodiment of the invention, the slide of the needle 21 in the sleeve 16 allows a correct alignment of the hooks provided in the upper part of the sleeve and of the corresponding hooking means provided on the flange of the plunger.

The invention has been shown with two hooks 16a and 16b formed at the level of those parts of the sleeve 16 of smallest radius of curvature. According to a variant embodiment of the invention (not shown), these hooks might be arranged at the level of those parts of largest radius of curvature of this sleeve, i.e. the parts shown respectively towards the top and towards the bottom of FIG. 3. In that case, the position of the openings 22a and 22b and of the surfaces 22d and 22e of the flange 22 with respect to the needle 21 is adapted accordingly. In addition, the number of hooks of the device of the invention is not limited to two; one hook is sufficient for it to immobilize the plunger in the position of transfer. In a variant embodiment, a number of hooks greater than or equal to three may be provided.

The invention has been shown with a one-piece plunger. It is also applicable to the case of the plunger being formed of an assembly comprising a needle and an added flange.

1. Device for connection between a closed recipient and a container, said closed recipient comprising a neck whose opening is closed by a stopper, said connection device comprising:
a base adapted to be mounted on said recipient and comprising a sleeve forming an inner bore (A), and
a plunger adapted to slide in said bore, between a first position disengaged with respect to said stopper and a second so-called position of transfer, in which a hollow needle belonging to said plunger, traverses said stopper,
wherein said needle presents a non-circular outer cross-section, while said sleeve presents a likewise non-circular inner cross-section, the outer section of said needle and inner section of said sleeve being such that said needle can slide in said sleeve, without the possibility of rotation of said needle in said sleeve, and
an edge of said sleeve opposite said stopper is provided with stop means adapted to cooperate with complementary means provided on said plunger to lock it in position of transfer.

2. The device of claim 1, wherein said needle presents an oval outer crosssection, while the inner cross-section said sleeve is oval.

3. The device of claim 1, wherein said needle presents a polygonal outer cross-section, while the inner cross-section of said sleeve is polygonal, with the same number of sides as said outer section of said needle.

4. The device of claim 1, wherein said stop means provided on said edge of said sleeve comprise elastically deformable hooks while said complementary means provided on said plunger comprise bearing surfaces made on a flange in one piece with said needle, said needle and said flange together constituting said plunger.

5. The device of claim 4, wherein said flange is provided with openings for passage of said hooks.

6. The device of claim 4, wherein said hooks are each provided with a nose adapted to be imbricated with a return bordering one of the bearing surfaces made on said flange.

7. The device of claim 4, wherein said hooks project radially towards the outside with respect to said sleeve.

8. The device of claim 1, wherein said base comprises a second sleeve disposed radially outside said sleeve, said second sleeve being adapted to cooperate with a cap for protecting said plunger with respect to the ambient atmosphere.

9. The device of claim 8, wherein it comprises rigidifying ribs connecting said sleeves.

10. The device of claim 1, wherein it comprises means for temporarily stopping said plunger in said disengaged position.

11. The device of claim 10, wherein said means comprise at least one hollow made on the outer surface of said needle and at least one projection extending, from the inner radial surface of said sleeve, in the direction of a central axis (X-X′) of said bore (A), said projection being adapted to be engaged in said hollow and to maintain said plunger in said first position.

12. Ready-to-use assembly comprising a closed recipient containing a product, particularly a pharmaceutical preparation, said recipient being provided with a neck whose opening is closed by a stopper, and a connection device according to claim 1, mounted on said recipient.
